SEQUENCE LISTING (16 sequences)

SEQ ID NO: 1 - DNA, 7354 bp, Artificial
  pHSVGET plasmid
gtcgagcaggactagtcttttaggtcaaaaagaagaagctttgtaaccgttggtttccgtagtgtagtggttatcacgttcgcctaacacgcgaaaggtccccggttcgaaggatcccagcttgaattcgactctagattttttggaatggttttttttttacaccacatcgtcgacccgggcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcaatttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcgctaccggtcgccaccatggtgagcaagggcgaggagctgttcaccggggtggtgcccatcctggtcgagctggacggcgacgtaaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggcgttcgtgaccgccgccgggatcactctcggcatggacgagctgtacaagtccggactcagatccaccggatctaggggcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcaattccagctgagcgccggtcgctaccattaccagttggtctggtgtcaaaaataataataaccgggcaggccatgtctgcccgtatttcgcgtaaggaaatccattatgtactatttaaaaaacacaaacttttggatgttcggtttattctttttcttttacttttttatcatgggagcctacttcccgtttttcccgatttggctacatgacatcaaccatatcagcaaaagtgatacgggtattatttttgccgctatttctctgttctcgctattattccaaccgctgtttggtctgctttctgacaaactcggaacttgtttattgcagcttataatggttacaaataaagcaatagcatcacaaatttcacaaataaagcatttttttcactgcattctatgtttatttcgttatcgtagtgtttaaagtgtttatttcgtaaaaaaagtgacgtaagatgttgtggttgtccaaactcatcaatgtatcttatcatgtctggatctctgacctgagattggcggcactgaggtagagatgcccgaacccccccgagggagcgcgggacgcgccggggagggctggggccggggagggctggggccggggagggctggggccggggagggctggggccggggagggctggggccggggagggctggggccggggagggctggggctggggagggctggggctggggagggggcggtggtgtgtagcaggagcggtgtgttgcgccggggtacgtctggaggagcgggaggtgcgcggtgacgtgtggatgaggaacaggagttgttgcgcggtgagttgtcgctgtgagttgtgttgttgggcgggtgtggtggatgacgtgacgtgtgacgtgcggagtgcgccgtgctctgttggtttcacctgtggcagcccgggccccccgcgggcgcgcgcgcgcgcaaaaaaggcgggcggcggtccgggcggcgtgcgcgcgcgcggcgggcgtggggggcggggccgcgggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggccagacgccgaaaacgggccccccccaaaacacaccccccgggggtcgcgcgcggccctttaaagcggtggcggcgggcagcccgggccccccgcggccgagactagcgagttagacaggcaagcactactcgcctctgcacgcacatgcttgcctgtcaaactctaccaccccggcacgctctctgtctccatggcccgccgccgccgccatcgcggcccccgccgcccccggccgcccgggcccacgggcgccgtcccaaccgcacagtcccaggtaacgagctcgaattaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcgcgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagvgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagccggccattatgcacgaccccgccccgacgccggcacgccgggggcccgtggccgcggcccgttggtcgaacccccggccccgcccatccgcgccatctgccatgggcggggcgcgagggcgggtgggtccgcgccccgccccgcatggcatctcattaccgcccgatccggcggtttccgcttccgttccgcatgctaacgaggaacgggcagggggcggggcccgggccccgacttcccggttcggcggtaatgagatacgagvvvvgcgcgcccgttggccgtccccgggccccccggtcccgcccgccggacgccgggaccaacgggacggcgggcggcccaagggccgcccgccttgccgcccccccattggccggcgggcgggaccgccccaagggggcggggccgccgggtaaaagaagtgagaacgcgaagcgttcgcacttcgtcccaatatatatatattattagggcgaagtgcgagcactggcgccgtgcccagctccgcgccggccccgggggcgggcccgggcggcggggggcgggtctctccggcgcacataaaggcccggcgcgaccgacgcccgcagacggcgccggccacgaacgacgggagcggctgcggagcacgcggaccgggagcgggagtcgcagagggccgtcggagcggacggcgtcggcatcgcgacgccccggctcgggatcgggatcgcatcggaaagggacacgcggacgcgggggggaaagacccgcccaccccacccacgaaacacaggggacgcaccccgggggcctccgacgacagaaacccaccggtccgccttttttgcacgggtaagcaccttgggtgggcggaggagggggggacgcgggggtggaggaggggggacgcgggggcggaggaggggggacgcgggggcggaggaggggggacgcgggggcggaggaggggggacgcgggggcggaggagggggctcacccgcgttcgtgccttcccgcaggaggaacgtcctcgtcgat

SEQ ID NO: 2 - DNA, 21 bp, Herpes simplex virus-1
ggcagcccgggccccccgcgg

SEQ ID NO: 3 - DNA, 21 bp, Herpes simplex virus-1
ccgcggggggcccgggctgcc

SEQ ID NO: 4 - DNA, 68 bp, artificial
  primer
ccctcgagcaggactagtcttttaggtcaaaaagaagaagctttgtaaccgttggtttccgtagtgta

SEQ ID NO: 5 - DNA, 71 bp, artificial
  primer
ccggatccttcgaaccggggacctttcgcgtgttaggcgaacgtgataaccactacactacggaaaccaac

SEQ ID NO: 6 - DNA, 21 bp, artificial
  APP-BP1 siRNA
ggtagatatccaggagtatct

SEQ ID NO: 7 - DNA, 59 bp, artificial
  primer
gatcggtagatatccaggagtatcttcaagagagatactcctggatatctacctttttt

SEQ ID NO: 8 - DNA, 59 bp, artificial
  primer
ctagaaaaaaggtagatatccaggagtatctctcttgaagatactcctggatatctacc

SEQ ID NO: 9 - DNA, 21 bp, artificial
  APP siRNA
gctgataagaaggcagttatc

SEQ ID NO: 10 - DNA, 59 bp, artificial
  primer
gatcgctgataagaaggcagttatctcaagaggataactgccttcttatcagctttttt

SEQ ID NO: 11 - DNA, 59 bp, artificial
  primer
ctagaaaaaagctgataagaaggcagttatcctcttgagataactgccttcttatcagc

SEQ ID NO: 12 - DNA, 55 bp, artificial
  APP-BP1 shRNA
ggtagatatccaggagtatcttcaagagagatactcctggatatctacctttttt

SEQ ID NO: 13 - DNA, 55 bp, artificial
  APP shRNA
gctgataagaaggcagttatctcaagaggataactgccttcttatcagctttttt

SEQ ID NO: 14 - DNA, 59 bp, artificial
  APP1996 shRNA sense strand
gatccgcagaagatgtgggttcaaactcaagaggtttgaacccacatcttctgcttttt

SEQ ID NO: 15 - DNA, 21 bp, artificial
  APP1996 siRNA
gcagaagatgtgggttcaaac

SEQ ID NO: 16 - DNA, 54 bp, artificial
  APP1996 shRNA
gcagaagatgtgggttcaaactcaagaggtttgaacccacatcttctgcttttt

(19) United States
(12) Patent Application Publication
(10) Pub. No.: US 2006/0154370 A1
(43) Pub. Date: Jul. 13, 2006

(54) Efficient gene suppression using a transfer RNA promoter in herpes virus vectors to deliver small interference RNAs

(76) Inventor: Yuzhi Chen, Little Rock, AR (US)

Correspondence Address: Hugh McTavish;McTavish Patent Firm, 429 Birchwood Courts, Birchwood, MN, 55110, US

(21) Appl. No.: 11/327,232

(22) Filed: Jan. 7, 2006

Related U.S. Application Data
(60) Provisional application No. 60/643,065, filed on Jan. 11, 2005.

(51) Int. Cl. C12N 15/86; C07H 21/04

(52) U.S. Cl. 435/456; 536/23.72

(57) ABSTRACT

The invention provides herpes virus nucleic acid vectors for expressing shRNAs in mammalian cells and thereby silencing target genes. The vectors include (a) a herpes virus packaging signal sequence; (b) a herpes virus origin of replication; (c) a segment expressing a light-emitting marker; and (d) a transfer RNA promoter upstream of a restriction endonuclease recognition sequence. A segment encoding an shRNA can be cloned into the restriction endonuclease recognition sequence. Thus, the invention also provides vectors containing: (a) a herpes virus packaging signal sequence; (b) a herpes virus origin of replication; (c) a segment expressing a light-emitting marker; and (d) a transfer RNA promoter linked to (e) a segment encoding a short hairpin RNA (shRNA) that is adapted to degrade in vivo to a small interference RNA (siRNA) that is complementary to a segment of a target gene.

This application claims priority from U.S. provisional patent application Ser. No. 60/643,065, Efficient Gene Suppression Using a Transfer RNA Promoter In Herpes Virus Vectors To Deliver Small Interference RNAs, filed Jan. 11, 2005.

### BACKGROUND

RNA interference has been shown to be an effective mechanism of gene silencing (7). Tuschl and colleagues (6) showed that transfection of synthetic 21-base-pair small interfering RNA (siRNA) duplexes into mammalian cells efficiently inhibits endogenous gene expression in a sequence-specific manner. These and other double-stranded RNAs complementary to mRNAs lead to cleavage of mRNA at sites 21-23 nucleotides apart (29). Without continuous production of the siRNAs in the cell, however, the inhibition of expression is short-lived.

Brummelkamp et al. (2) reported the use of plasmids in mammalian cells that express short hairpin RNAs similar to the double-stranded siRNA. The shRNAs inhibited target gene expression. The shRNA included a 19-nt sequence derived from the target transcript, separated by a short spacer sequence (e.g., 6-nt) from the reverse complement of the same 19-nt sequence. The resulting RNA transcript is predicted to fold into a 19-base-pair stem loop structure. The shRNA was transcribed from an H1 RNA polymerase III promoter (2). Paddison et al. reported that shRNAs of about 70 nt in length and having a 22-29 base-pair (bp) stem loop stucture when added directly to cells inhibited expression of a target mRNA complementary to one strand of the 22-29 base-pair stem (22). They also reported cloning an shRNA-encoding sequence behind a U6 polymerase III promoter in a plasmid to silence a luciferase target gene (22). In that case the shRNA had a 29-bp stem loop structure (22).

Short hairpin RNAs are thought to be processed by the Dicer enzyme into siRNAs that hybridize to the mRNA of the target gene, inducing degradation of the mRNA (1, 12).

Some of the more difficult cells to genetically modify are neuronal cells. Neurons are postmitotic, and so cannot be stably transformed with vectors that depend on cell division for their maintenance. Retroviral vectors may not depend on cell division for maintenance, but they integrate into the host cell genome, which can disrupt gene expression at the site of integration. New tools and methods for inhibiting target gene expression in neurons are needed. These will be useful for studying gene function in neurons, screening for proteins that would be suitable drug targets in neurons, and treating diseases of the brain and nervous system.

### SUMMARY

The invention provides a herpes virus-based vector that expresses a light-emitting marker such as green fluorescent protein (GFP), and that contains a transfer RNA promoter, preferably the tRNA-valine promoter. The tRNA promoter is preferably immediately upstream of a restriction site, into which a sequence encoding a short hairpin RNA designed to silence expression of a target gene in a cell can be inserted. The herpes virus-based vector is preferably a herpes simplex virus 1 (HSV-1) vector. Herpes virus vectors infect many mammalian cell types including non-dividing cells such as neurons. They infect cells efficiently. And they can persist in neurons indefinitely. A light-emitting marker such as GFP allows easy identification of infected cells, and allows easy quantification of the amount of vector in a cell, so that the titer of the vector is easily determined. Short hairpin RNA transcripts of the tRNA-valine promoter are efficiently transcribed with accurate and consistent start and end points. The shRNA transcripts of the tRNA-valine promoter are transported to the cytoplasm, where they are efficiently processed by Dicer to generate siRNAs that silence the target gene by binding to and causing the degradation of the mRNA transcript of the target gene (12). It is shown here that these vectors efficiently infect neurons in vitro and efficiently silence target genes.

Accordingly, one embodiment of the invention provides a recombinant nucleic acid molecule containing: (a) a herpes virus packaging signal sequence; (b) a herpes virus origin of replication; (c) a segment expressing a light-emitting marker; and (d) a transfer RNA promoter upstream of a restriction endonuclease recognition sequence.

Another embodiment of the invention provides a recombinant nucleic acid molecule containing: (a) a herpes virus packaging signal sequence; (b) a herpes virus origin of replication; (c) a segment expressing a light-emitting marker; and (d) a transfer RNA promoter linked to (e) a segment encoding a short hairpin RNA (shRNA) that is adapted to degrade in vivo to a small interference RNA (siRNA) that is complementary to a segment of a target gene.

Another embodiment of the invention is herpes virus particles containing a recombinant nucleic acid molecule having: (a) a herpes virus packaging signal sequence; (b) a herpes virus origin of replication; (c) a segment expressing a light-emitting marker; and (d) a transfer RNA promoter upstream of a restriction endonuclease recognition sequence.

Another embodiment of the invention is herpes virus particles containing a recombinant nucleic acid molecule having: (a) a herpes virus packaging signal sequence; (b) a herpes virus origin of replication; (c) a segment expressing a light-emitting marker; and (d) a transfer RNA promoter linked to (e) a segment encoding a short hairpin RNA (shRNA) that is adapted to degrade in vivo to a small interference RNA (siRNA) that is complementary to a segment of a target gene.

Another embodiment of the invention provides a method of inhibiting expression of a target gene in cells involving (i) transforming the cells with a recombinant nucleic acid molecule that includes a segment encoding a short hairpin RNA that is adapted to degrade in vivo to a small interference RNA (siRNA) that is complementary to a segment of a target gene; and (ii) expressing the shRNA in the cell. The recombinant nucleic acid molecule includes (a) a herpes virus packaging signal sequence; (b) a herpes virus origin of replication; (c) a segment expressing a light-emitting marker; and (d) a transfer RNA promoter linked to the segment encoding the shRNA.

Another embodiment of the invention provides a method of treating a neuronal disease in a mammal involving: (i) transforming neurons in the mammal with herpes virus particles containing a recombinant nucleic acid molecule containing: a segment encoding a short hairpin RNA (shRNA) that is adapted to degrade in vivo to a small interference RNA (siRNA) that is complementary to a segment of a target gene whose expression promotes the disease; and (ii) expressing the shRNA in the neurons to decrease expression of the target gene. The recombinant nucleic acid molecule includes (a) a herpes virus packaging signal sequence; (b) a herpes virus origin of replication; (c) a segment expressing a light-emitting marker; and (d) a transfer RNA promoter linked to the segment encoding the shRNA.

Another embodiment of the invention is a cell, e.g., a mammalian cell, containing one of the recombinant nucleic acid molecules of the invention. The cell can be a eukaryotic or a prokaryotic cell, e.g., a yeast cell or E. coli cell.

Another group of embodiments of the invention is particular siRNAs and shRNAs, and nucleic acid molecules encoding them, that inhibit amyloid precursor protein (APP) and APP binding protein (APP-BP1). APP and APP-BP1 are two proteins implicated in Alzheimer's disease. One embodiment of the invention is a recombinant nucleic acid comprising 5′-GGTAGATATCCAGGAGTATCT-3′ (SEQ ID NO:6), which is an siRNA against APP-BP1. Another embodiment is a recombinant nucleic acid comprising 5′-GCTGATAAGA AGGCAGTTAT C-3′ (SEQ ID NO:9), which is an siRNA against APP. Another embodiment of the invention is a recombinant nucleic acid comprising 5′-GCAGAAGATGTGGGTTCAAAC-3′ (SEQ ID NO:15), which is another siRNA against APP, designated APP 1996 siRNA.

### BRIEF DESCRIPTION OF THE DRAWINGS

FIG. 1. Western blot showing that APP-BP1 shRNA suppresses specific gene expression in primary neurons. Herpes virus vector expressing APP-BP1 shRNA or random sequence missense shRNA infected rat primary neurons at 0.5 or 1 IU per cell. Infection lasted for 14 hours before cell lysis. Protein from total lysates was resolved on an 8% SDS-PAGE gel and transferred to nitrocellulose membrane, which was probed with BP339, a rabbit polyclonal antibody against APP-BP1. Gamma-tubulin was used as a control in the western blots.

FIG. 2. Western blot showing APP shRNA virus suppressed APP expression in rat primary neurons. Herpes virus vectors expressing APP shRNA or a random sequence negative control shRNA were used at 0.5 IU per cell for infection. Proteins were analyzed on a 12% SDS-PAGE gel and blotted, and the blot was probed with anti-APP antibody.

FIG. 3. Western blot showing rat endogenous APP was suppressed by APP shRNA virus APP1996 at 1 IU per cell in primary neuronal culture. Neurons were infected for 14 hours before lysis and protein analysis.

FIG. 4. Bar graph showing expression levels of Aβ42 and Aβ40 in neurons expressing APP shRNA or APP-BP1 shRNA. Suppression of APP-BP1 protein expression by APP-BP1 shRNA results in a strong increase of intracellular Aβ42. Intracellular (from 50 μg protein) and secreted (from 1/15 volume of conditioned medium) Aβ42 in primary neurons were determined by ELISA (left). Intracellular (from 50 μg protein) and secreted (from 1/30 volume of conditioned medium) Aβ40 in primary neurons were determined by ELISA (right). Cells expressed APP695 from an HSV-1 virus vector, and were transformed in addition with no vector, APP shRNA virus, APP-BP1 shRNA virus, or the random shRNA virus. The amount of Aβ in samples that expressed APP without any shRNA interference was used as 100% to normalize. Data is representative of two independent experiments.

FIG. 5. Western blot showing APP-BP1 siRNA expression was associated with increases in APP C-terminal fragment levels in neuronal lysates. Equal amounts of totol protein from cell lysate were analysed on a 16% Tris-tricine gel and blotted to detect APP C-terminal fragment (CTF) using rabbit polyclonal antibody 369 raised against amino acids 645-694 of APP695. A positive control was cell lysates prepared from non-infected cells treated with the gamma-secretase inhibitor L685459. The first three lanes were from samples expressing APP695. CHEMIGLOW from Alpha Innotech was used for the chemiluminescence reaction.

### DETAILED DESCRIPTION

Definitions:

The term “small interference RNA” refers to a double-stranded RNA molecule of about 17 to about 29 base pairs in length, one strand of which is complementary to a target mRNA, that when added to a cell having the target mRNA or produced in the cell in vivo, causes degradation of the target mRNA. Preferably the siRNA is perfectly complementary to the target mRNA. But it may have one or two mismatched base pairs. The term “siRNA” is also sometimes used herein to refer to the single strand of a double-stranded siRNA that is complementary to a target mRNA, as will be clear from the context.

The term “short hairpin RNA” as used herein refers to an RNA molecule that forms a stem-loop structure in physiological conditions, with a double-stranded stem of about 17 to about 29 base pairs in length, where one strand of the base-paired stem is complementary to the mRNA of a target gene. The loop of the shRNA stem-loop structure may be any suitable length that allows inactivation of the target gene in vivo. Preferably the loop is 3-30 nucleotides in length. More preferably it is 3-9 nucleotides in length (28). The base paired stem may be perfectly base paired or may have 1 or 2 mismatched base pairs. Preferably the stem is perfectly base paired. The shRNA may have non-base-paired 5′ and 3′ sequences extending from the base-paired stem. Typically, however, there is no 5′ extension. The first nucleotide of the shRNA at the 5′ end is a G, because this is the first nucleotide transcribed by polymerase III. If G is not present as the first base in the target sequence, a G may be added before the specific target sequence. The 5′ G typically forms a portion of the base-paired stem. Typically, the 3′ end of the shRNA is a poly U segment that is a transcription termination signal and does not form a base-paired structure.

The term “herpes virus packaging signal sequence” is a nucleotide sequence found in the herpes virus genome, or a sequence homologous to such a sequence, that is necessary for a DNA molecule to be packaged by herpes virus proteins into herpes virus particles. If the packaging signal sequence is only homologous to a native herpes virus packaging signal sequence, preferably it is at least 90% identical to a native herpes virus packaging signal sequence.

The term “origin of replication that functions in a mammalian cell” as used herein refers to a nucleotide sequence that allows replication of an episomal nucleic acid molecule in a mammalian cell and that includes the point at which DNA replication of the nucleic acid molecule initiates in vivo in the mammalian cell.

A “herpes virus origin of replication” as used herein is a nucleotide sequence found in a herpes virus that is necessary for replication of the herpes virus genome and that includes a point at which DNA replication is initiated in the herpes virus, or a sequence homologous to the native sequence that can function to support replication of a recombinant herpes virus and includes a point at which replication is initiated. Preferably if the origin of replication is only homologous to a native herpes virus origin of replication, it is at least 90% identical to the sequence of a native herpes virus origin of replication.

The term “tRNA promoter” as used herein refers to a nucleotide sequence found in nature as a promoter for transcription of a transfer RNA in a mammal. The term also includes a nucleotide sequence that is at least 90% identical to a native tRNA promoter sequence and that is able to support transcription of a transfer RNA in a recombinant system in a mammalian cell.

Nucleic acid sequences given herein contain T to denote thymidine. It is understood that in RNA molecules corresponding to these sequences, the Ts are replaced with Us (uridines).

Description:

The vectors of the invention are used to express shRNAs that are processed in a cell to siRNAs complimentary to a target gene. The siRNA can hybridize to the mRNA of the target gene and thereby silence or reduce expression of the target gene. The HSV vectors are particularly suited to use to inhibit expression of target genes in neurons, and thus suited for investigation and possible treatment of neuronal diseases through silencing target genes implicated in neuronal diseases.

The vectors have been used to silence genes implicated in Alzheimer's disease (AD). Among the genes implicated in AD are the genes encoding amyloid precursor protein (APP) and tau. AD is a progressive dementia associated with certain neurological lesions including extracellular deposits of aggregated amyloid β (Aβ) proteins, which are proteolytically derived from APP, and intracellular neurofibrillary tangles in the brain (31). Tau is the major component of the neurofibrillary tangles (20).

APP is a transmembrane receptor protein (31). Overexpression of APP in primary neurons induces neuronal apopotosis (31). Down syndrome or trisomy 21 is characterized by early onset AD, and Down syndrome patients have an extra copy of the APP gene with their third copy of chromosome 21 (31). This suggests, along with the finding that overexpressions of APP induces apoptosis of primary neurons (31), that overexpression of wild type APP may lead to AD. Several mutant forms of APP have also been linked to AD, including APPsw (20, 32). A mutant form of tau, tauV337M, has also been linked to AD and other neurological diseases (20, 33-35).

Another protein possibly involved in AD is the APP binding protein-1 (APP-BP1) (31). APP-BP1 was identified as a protein that interacts with the cytoplasmic domain of APP (31). APP-BP1 was determined to be the regulatory subunit for the NEDD8 activating enzyme (36). APP-BP1 drives the S to M transition in dividing cells and causes apoptosis in neurons (36, 37).

Certain embodiments of the invention involve methods to inhibit the expression of APP, tau, or APP-BP1, including mutants thereof, in neurons or other cells by expressing an shRNA in the cells.

The genes for presenilin 1 and 2 are also genetically linked to AD. Certain mutant forms of presenilin 1 and 2 cause increases in Aβ-42, a form of amyloid β. Inhibiting the expression of wild-type presenilin 1 and 2 probably would not be beneficial, because the presenilins process by proteolytic cleavage many transmembrane proteins. But inhibiting expression of the mutant forms of presenilin 1 and 2 by expressing shRNAs may be beneficial as a possible treatment for AD.

The nucleic acid molecules of the invention can be engineered full-length herpes virus vectors containing the bulk of the herpes virus genome. The wild-type HSV-1 genome is approximately 150 kb, so this type of vector would approach that size. More preferably, the nucleic acid molecules of the invention are much smaller amplicons, having only a small number of herpes virus genes, such as the packaging signal sequence and the herpes virus origin of replication. No other herpes virus sequences need to be included in the vector, although they can be. The smaller vectors are termed plasmids or amplicons. They can be, for instance, 5-10 kb. They may be packaged into herpes virus particles with coinfection of a helper virus or in cells harboring cosmids that provide packaging functions in trans (15, 21).

In the recombinant nucleic acid molecules of the invention having a tRNA promoter upstream of a restriction endonuclease recognition sequence, the tRNA promoter is functionally linked to the restriction endonuclease recognition sequence. That is, a sequence inserted into the restriction site can be transcribed from the promoter. Preferably, the restriction site is within 100 bp, preferably within 20 bp, most preferably within about 10 bp of the promoter.

Preferably the restriction endonuclease recognition sequence is a 6-base pair sequence. It may be alternatively be, e.g., an 8-bp sequence. A 4-bp recognition sequence would be less preferable since such a sequence is likely to be found elsewhere in the vector. Preferably the restriction site or sites for cloning linked to the tRNA promoter are found only once in the vector.

The origin of replication for the recombinant nucleic acid molecules is a herpes virus origin of replication. In preferred embodiments it is an HSV-1 origin of replication.

In certain embodiments, the HSV-1 origin of replication is or includes the HSV-1 OriS core region, which is nucleotides 6651-6849 of SEQ ID NO:1. (SEQ ID NO:1 is an example of a vector of the invention.) In some embodiments, the HSV-1 origin of replication is or includes the full OriS (18), nucleotides 6334-7107 of SEQ ID NO:1. It has been shown that the flanking regions are not strictly essential to function of the OriS core, but increase its activity in a plasmid vector as much as 80 fold (26b). But the flanking regions could be replaced with heterologous sequences, such as the cytomegalovirus immediate-early promoter (26b).

The vectors may include an additional origin of replication that functions in a mammalian cell, such as another viral origin of replication, e.g., an adeno-associated virus origin of replication. The vectors may also include a bacterial origin of replication to allow manipulation of the vector in E. coli or another bacterium.

In preferred embodiments, the packaging signal sequence is an HSV-1 packaging signal sequence. An example of a minimal HSV-1 packaging signal sequence is nucleotides 3418-3438 of SEQ ID NO:1, which is 5′-GGCAGCCCGGGCCCCCCGCGG-3′ (SEQ ID NO:2), or its complement 5′-CCGCGGGGGGCCCGGGCTGCC-3′ (SEQ ID NO:3) (reference 5). SEQ ID NO:2 is also found at nucleotides 3817-3837 of SEQ ID NO:1. The complete alpha sequence (packaging signal sequence, reference 24) of HSV-1 is nucleotides 3011-4021 of SEQ ID NO:1. Thus, in a particular embodiment, the packaging signal sequence includes SEQ ID NO:2, or its complement. In another particular embodiment, the packaging signal sequence includes nucleotides 3011-4021 of SEQ ID NO:1, or its complement.

In preferred embodiments, the transfer RNA promoter is a tRNA^{val} promoter.

In preferred embodiments, the transfer RNA promoter is a human transfer RNA promoter, e.g., a human tRNA^{val} promoter. In a particular embodiment, the tRNA^{val} promoter is or includes nucleotides 7-113 of SEQ ID NO:1.

In preferred embodiments, the light-emitting marker is green fluorescent protein (GFP). The term “green fluorescent protein” or “GFP” as used herein includes enhanced GFP and other variant forms of GFP. An example of a GFP-encoding sequence is nucleotides 1507-2250 of SEQ ID NO:1.

In other specific embodiments, the light-emitting marker may be luciferase. Emission of light from luciferase requires oxygen, ATP, and the cofactor luciferin. Thus, if the light emitting marker is luciferase it is typically necessary to add luciferin to the cells transformed with the nucleic acid in order to generate light.

In particular embodiments of the invention, the recombinant nucleic acid molecules are smaller than 15 kb. Plasmid or amplicon vectors of the invention are typically smaller than 15 kb.

In other embodiments, the recombinant nucleic acid molecules of the invention are at least 15 kb in size. Defective virus vectors are typically close to the wild type herpes virus size and are much larger than 15 kb, e.g., approximately 150 kb.

In particular embodiments, the recombinant nucleic acid molecule includes herpes virus nucleic acid sequences other than the packaging signal sequence and a herpes virus origin of replication. Defective HSV-1 virus vectors, for instance, include the majority of the HSV-1 genome.

In particular embodiments of the invention, the shRNA encoded by the nucleic acid forms a stem-loop structure having a stem of 19 to 29 base pairs. More preferably, the stem is 19 to 25 base pairs, more preferably still 19 to 23 base pairs, and most preferably about 21 base pairs. Polymerase III transcribes short mRNAs accurately and efficiently (10).

The unpaired loop of the shRNA encoded by the nucleic acid molecules of the invention is preferably about 3 to 9 nucleotides long, but may be any size that allows the shRNA to generate an siRNA in vivo that inhibits expression of the target gene (28).

The vectors of the invention may be hybrid vectors that include at least one segment of viral nucleic acid from a non-herpes virus. For instance, they may include the Epstein-Barr virus segments oriP and EBNA-1 (26). A hybrid herpes virus vector containing Epstein-Barr virus segments oriP and EBNA-1 is described in reference 26. Those two segments allow vector episomal maintenance in some cells and can assist in generating viral stocks of high titer (26).

The vectors of the invention in some embodiments are hybrid vectors containing at least two adeno-associated virus (AAV) terminal repeats (11). The AAV terminal repeats may flank the expression cassette containing the tRNA promoter linked to a restriction site or the shRNA-encoding sequence (11). This facilitates replication and integration of the cassette in the host cell nucleus (11).

One embodiment of the invention is herpes virus particles containing the recombinant nucleic acid molecules of the invention. In a preferred embodiment, the virus particles are HSV-1 particles—i.e., they include HSV-1 capsid proteins.

The virus particles may be prepared by a process involving contacting host cells with the recombinant nucleic acid molecule and with a herpes virus deletion mutant. Detailed protocols for preparing herpes virus vector stocks with deletion mutant helper viruses are provided in reference 15. One suitable HSV-1 helper virus is D30EBA (9, 23). Other suitable HSV-1 helper viruses include those with deletions in the IE3 gene, such as 5dl1.2 (14).

Herpes virus particles containing the recombinant nucleic acid molecules of the invention can also be prepared by a helper-virus-free process. For packaging recombinant molecules containing the HSV-1 packaging signal in HSV-1 particles, the process can involve contacting host cells with a recombinant nucleic acid molecule of the invention and harvesting viral particles produced by the host cells; wherein the host cells carry one or more other recombinant nucleic acid molecules (packaging nucleic acid molecules) collectively containing most of the HSV-1 genome and lacking HSV-1 DNA cleavage/packaging signals. In this case, the vector nucleic acid molecule and the packaging nucleic acid molecules can simultaneously cotransform the host cells, or they can transform the host cells in any order. In a particular embodiment, the host cells carry the cosmid set C6Δa48Δa (8).

One embodiment of the invention involves a method of inhibiting expression of a target gene in cells involving transforming cells with a vector of the invention containing a tRNA promoter linked to a segment encoding an shRNA directed to the target gene, and expressing the shRNA in the cell.

In particular embodiments, the cells are neuronal cells.

In preferred embodiments, the cells transformed with the vectors are post-mitotic cells, e.g., muscle cells or neuronal cells (38).

The cells may be transformed in vivo in a mammal or in vitro.

The cells may be transformed in vitro and then implanted into a mammal.

In particular embodiments, the shRNA is expressed in vivo in a mammal to inhibit expression of the target gene.

In preferred embodiments, the recombinant nucleic acid to transform the cells is encased in herpes virus capsid proteins to form herpes virus particles, and the cells are transformed with the virus particles. Cells may also be transformed with naked recombinant nucleic acids of the invention.

One of the advantages of having a segment expressing GFP or another light-emitting marker in the vectors is that it allows fast and easy titration of the amount of infectious particles or the amount of vector. Cells are infected with the virus particles (or transformed with naked vector) and then the number of cells emitting light or the amount of light emission (e.g., from GFP) is determined, e.g., by fluorescence microscopy.

Thus, in one embodiment of the method of inhibiting target gene expression in host cells, the cells are transformed with a known quantity of virus particles, wherein the quantity is determined by titrating the virus particles by transforming cells with the virus particles and measuring light emitted (e.g., quantifying total light emitted or quantifying the number of cells emitting light).

In another embodiment of the method of inhibiting target gene expression in host cells, the cells are transformed with a known quantity of a recombinant nucleic acid molecule of the invention, wherein the quantity is determined by titrating the recombinant nucleic acid molecules by transforming cells with the recombinant nucleic acid molecules and measuring light emitted (e.g., quantifying total light emitted or quantifying the number of cells emitting light).

In specific embodiments of the method of inhibiting target gene expression, the target gene is an APP, APP-BP1, or tau gene.

In particular embodiments, the target gene may be a mutant form of an APP gene or tau gene associated with Alzheimer's disease, e.g., APPsw (20, 32) or tauV337M (20, 33-35).

In particular embodiments, the target gene is a mutant form of presenilin 1 or 2.

To target a mutant gene differentially from a wild-type gene, an shRNA should be designed to generate an siRNA having nucleotides specific for the mutant form of the target gene located near the center of the siRNA, e.g., at approximately nuleotides 9-13 of a 21-nucleotide siRNA (20).

Another embodiment of the invention is a method of treating a neuronal disease in a mammal by transforming neurons in the mammal with herpes virus particles containing a recombinant nucleic acid of the invention expressing an shRNA, and expressing the shRNA in the neurons to decrease expression of a target gene.

For instance, the disease may be brain cancer. Inhibiting expression of target genes that promote survival of cancer cells with shRNA expression may be effective to treat brain cancer. Such target genes include PLK1 (25), p53 (27), survivin (16), and the IGF-1 receptor.

In another embodiment, the neuronal disease is Alzheimer's disease. This may be treated by inhibiting the target genes tauV337M or APPsw with shRNA from a vector of the invention (20).

Another group of embodiments of the invention is particular siRNAs and shRNAs, and nucleic acid molecules encoding them, that inhibit amyloid precursor protein (APP) and APP binding protein (APP-BP1). One embodiment of the invention is a recombinant nucleic acid comprising 5′-GGTAGATATCCAGGAGTATCT-3′ (SEQ ID NO:6), which is an siRNA against APP-BP1, or the complement thereof. Another embodiment is an shRNA that produces SEQ ID NO:6 as an siRNA, namely the shRNA 5′-GGTAGA TATCCAGGAG TATCTTCAAG AGAGATACTC CTGGATATCT ACCTTTTTT-3′ (SEQ ID NO:12). (See Example 2 below.) Thus, one embodiment of the invention is a recombinant nucleic acid comprising SEQ ID NO:12, or the complement thereof.

Another embodiment of the invention is a recombinant nucleic acid comprising 5′-GCTGATAAGA AGGCAGTTAT C-3′ (SEQ ID NO:9), which is an siRNA against APP, or the complement thereof. A more specific embodiment is a recombinant nucleic acid comprising 5′-GCTGAT AAGAAGGCAG TTATCTCAAG AGGATAACTG CCTTCTTATC AGCTTTTTT-3′ (SEQ ID NO:13), or the complement thereof. SEQ ID NO:13 is an shRNA that produces the siRNA SEQ ID NO:9 (Example 2 below).

Another embodiment of the invention is a recombinant nucleic acid comprising 5′-GCAGAAGATGTGGGTTCAAAC-3′ (SEQ ID NO:15), which is an siRNA against APP, or the complement thereof. A more specific embodiment is a recombinant nucleic acid comprising 5′-GCAGAAGATGTGGGTTCAAAC TCAAGAGGTT TGAACCCACA TCTTCTGCTT TTT-3′ (SEQ ID NO:16) or the complement thereof. SEQ ID NO:16 is an shRNA that produces the siRNA SEQ ID NO:15 (Example 2 below).

One embodiment of the invention provides a method of inhibiting expression of amyloid precursor protein binding protein-1 (APP-BP1) in cells involving: first, transforming the cells with a recombinant nucleic acid molecule comprising: (a) a promoter linked to (b) a segment encoding a short hairpin RNA (shRNA) that is adapted to degrade in vivo to a small interference RNA (siRNA) that is complementary to a segment of an APP-BP1 gene; and second, expressing the shRNA in the cells.

In specific embodiments, the recombinant nucleic acid molecule further comprises a herpes virus packaging signal sequence and a herpes virus origin of replication.

In particular embodiments, the recombinant nucleic acid molecule further comprises a segment encoding a light-emitting marker.

In specific embodiments, the promoter is a transfer RNA promoter.

The invention will now be illustrated by the following examples, which are intended to illustrate the invention but not limit the scope thereof.

### EXAMPLES

### Example 1

Construction of the shRNA Vector pHSVGET.

To clone the tRNA-valine (tRNA^{val}) promoter (12, 20) into the Xho1/BamHI sites in pGE-1 vector (from Stratagene), the following primers were synthesized:

(tRNA-F; SEQ ID NO:4)

| | |
|---|---|
| Forward | 5′-CCCTCGAGCA GGACTAGTCT TTTAGGTCAA |
| | AAAGAAGAAG CTTTGTAACC GTTGGTTTCC |
| | GTAGTGTA-3′ |
| and | |

(tRNA-R; SEQ ID NO:5)

| | |
|---|---|
| Reverse | 5′-CCGGATCCTT CGAACCGGGG ACCTTTCGCG |
| | TGTTAGGCGA ACGTGATAAC CACTACACTA |
| | CGGAAACCAA C-3′. |

The primers were annealed to each other, digested with XhoI and BamHI (the restriction sites are underlined), and ligated to the XhoI/BamHI-digested pGE-1. The resulting plasmid was named pGET. pGET was digested with Xho1/Sal1 to generate an approximately 370 bp fragment containing the tRNA^{val} promoter and BamHI/XbaI shRNA cloning sites. The fragment was ligated into the HSV vector digested with Sal1. The HSV vector was essentially as described by Clark et al. (4). It is an HSV-1 packaging vector containing the eGFP open reading frame. The resulting plasmid was an HSV-1 packaging vector, and was named pHSVGET (SEQ ID NO:1).

To clone a particular shRNA sequence into pHSVGET, DNA oligonucleotides encoding the shRNA and having BamHI and XbaI compatible ends are annealed into BamHI/XbaI-cut pHSVGET. Positive shRNA clones are first screened by colony PCR using a promoter-specific primer. Positive clones identified this way are then sequenced. The shRNA construct was then packaged into HSV-1 virus using 2-2 vero cell line and a replication-defective helper virus, 5dl1.2, according to reference 15. The shRNA virus is released from cells by freeze-thaw and sonication. The crude virus preparation is purified by centrifugation on a discontinuous sucrose gradient (15).

After the shRNA virus is packaged and purified, the titer (concentration of viral stock) is determined by infecting rat embryonic cortical neurons in culture with various dilutions of the viral stock, and determining whether cells are infected by examining the neurons for eGFP fluorescence using a fluorescence microscope.

### Example 2

### Use of HSV-1 shRNA Amplicons to Reduce Expression of Amyloid Precursor Protein and Binding Protein in Neurons

Introduction: Alzheimer's disease is characterized by two brain anatomical pathologies: senile plaques, which contain beta-amyloid derived from cleavage of amyloid precursor protein (APP), and neurofibrillary tangles, which contain filamentous tau protein. In this study, the processing of APP is characterized by use of shRNAs directed against APP or APP binding protein (APP-BP1).

Methods and Results:

A DNA encoding an shRNA to target the amyloid precursor protein binding protein (APP-BP1) was designed to generate the siRNA 5′-GGTAGATATCCAGGAGTATCT-3′ (SEQ ID NO:6). The shRNA was designed with the Invitrogen online shRNA design program, BLOCK-IT™ RNAi Designer. The whole sequence of the sense strand for APP-BP1 shRNA cloning is 5′-GATCGGTAGA TATCCAGGAG TATCTTCAAG AGAGATACTC CTGGATATCT ACCTTTTTT-3′(SEQ ID NO:7). The antisense strand for APP-BP1 shRNA cloning is 5′-CTAGAAAAAA GGTAGATATC CAGGAGTATC TCTCTTGAAG ATACTCCTG GATATCTACC-3′ (SEQ ID NO:8). The predicted siRNA and its reverse complement, which together form the stem of the stem-loop shRNA structure are underlined in the sense strand. The shRNA-encoding segment was cloned into pHSVGET as described in Example 1. The amplicon was packaged into virus particles as described in Example 1.

Likewise, a DNA encoding an shRNA to target the amyloid precursor protein (APP) was designed to generate the siRNA 5′-GCTGATAAGA AGGCAGTTAT C-3′ (SEQ ID NO:9). The whole sequence of the sense strand for APPshRNA cloning is 5′-GATCGCTGAT AAGAAGGCAG TTATCTCAAG AGGATAACTG CCTTCTTATC AGCTTTTTT-3′ (SEQ ID NO:10). The antisense strand for APPshRNA cloning is 5′-CTAGAAAAAA GCTGATAAGA AGGCAGTTAT CCTCTTGAGA TAACTGCCTT CTTATCAGC-3′ (SEQ ID NO:11). The predicted siRNA and its reverse complement, which together form the stem of the stem-loop shRNA structure are underlined in the top strand. The shRNA-encoding segment was cloned into pHSVGET as described in Example 1. The amplicon was packaged into virus particles as described in Example 1.

Primary neurons for the titration of the virus and for experimental assays were plated at 2 to 2.5×10⁵ per cm² density in poly-D-lysine-coated plates and grown in Neural Basal Medium plus B27 supplements (Invitrogen), 1% fetal bovine serum, 1% equine serum, and 1× of penicillin/streptomycin (Sigma).

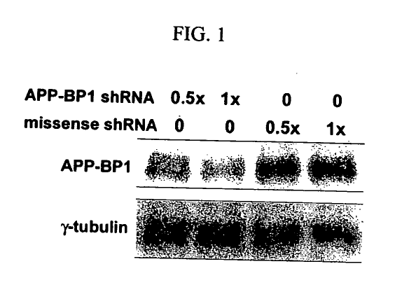

For titration of the viral stocks, primary neurons were infected with serially diluted viruses. For the experiment, primary neurons were infected at 1 infectious unit (IU) per cell of a vector expressing human APP-BP1. The cells infected with the APP-BP1-expressing vector were also infected at 1 or 0.5 infectious unit per cell with APP-BP1 siRNA virus, or with a virus vector carrying a 21-base-pair random sequence shRNA-encoding sequence (missense siRNA). Protein lysates from the cells were resolved on a 7.5% SDS-PAGE gel and transferred to nitrocellulose membrane, which was probed with BP339, a rabbit polyclonal antibody against APP-BP1 (FIG. 1). The results show that the amplicon encoding the APP-BP1 shRNA reduced expression of APP-BP1, while the vector expressing the missense siRNA did not.

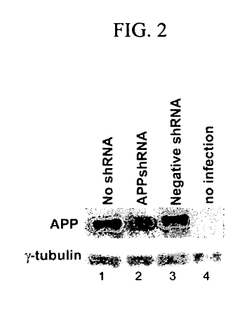

Primary neurons were also infected with 1 IU per cell of a vector expressing human APP695 (a human brain isoform of APP) (APP695 HSV). The cells infected with the APP695-expession vector were also infected with 0.5 IU per cell of virus containing pHSVGET expressing APP shRNA (SEQ ID NO:10) or the 21-base-pair random shRNA (Negative shRNA). The vector expressing APP shRNA reduced expression of APP while the negative shRNA did not (FIG. 2). APP protein was probed with the 369 antibody (gift from S. Gandy).

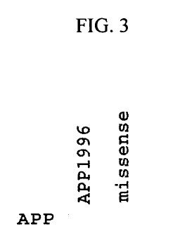

In another experiment, rat primary neurons were infected with 1 IU per cell of virus containing pHSVGET expressing an APP shRNA designated APP1996 or the 21-base-pair random siRNA (missense). APP1996 shRNA is encoded by the sequence 5′-GATCC GCAGAAGATGTGGGTTCAAAC TCAAGAGGTT TGAACCCACA TCTTCTGCTT TTT-3′ (SEQ ID NO:14). The underlined portion of SEQ ID NO:14 is the siRNA to be generated by the shRNA. This shRNA is designed to suppress endogenous rat or human APP. Cell lysates of the neurons were anaylzed by SDS-PAGE and Western blotting. The Western blot stained with anti-APP antibody is shown in FIG. 3. APP1996 was found to decrease endogenous rat APP expression as compared to the missense shRNA (FIG. 3).

Non-specific cytotoxic effects were observed if the primary neurons were infected at a higher multiplicity of infection with the APP shRNA or APP-BP1 shRNA vectors. The best specific results were obtained at 0.5 or 1 IU per cell. Cytotoxic effect was also noted with the lentivirus-mediated siRNA delivery vector (30).

The APP and APP-BP1 shRNAs did not induce an interferon-γ (INF-γ) response. No intracellular or secreted INF-γ was detected by INF-γ ELISA (Biosource International) (data not shown). This indicates that the reduced protein expression observed with the shRNA vectors is specific for the targeted genes and is not caused by stress-induced global shutdown of gene transcription. Confirming this interpretation, tubulin expression was constant in all cases (FIGS. 1 and 2).

To test the effect of APP-BP1 on production of beta-amyloid 40 and beta amyloid 42 (Aβ40 and Aβ42), neurons were infected for 16 hours with APP695 HSV-1, along with APP-BP1 shRNA virus, APP shRNA virus (SEQ ID NO:13), or a vector carrying the random shRNA (negative control), each at 1 IU per cell. Intracellular (from 50 μg of protein) and secreted (from 1/15 or 1/30 volume of medium) Aβ40 and Aβ42 were determined by ELISA.

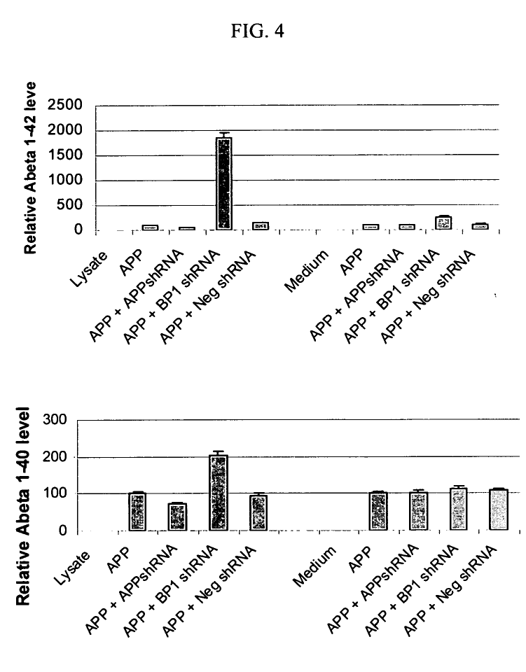

When the endogenous APP-BP1 of the neurons was suppressed with APP-BP1 shRNA, the intraneuronal Aβ42 was increased 19-fold (FIG. 4). Intraneuronal Aβ40 was increased to a lesser extent (FIG. 4). Aβ40 and Aβ42 were increased in the medium to a lesser degree than in the cytoplasm (FIG. 4). As a control, cells were infected with APP shRNA virus. This somewhat decreased Aβ40 and Aβ42 levels.

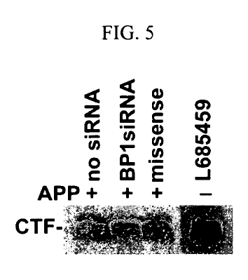

Immunoblot analysis of lysates from primary neurons expressing APP-BP1 shRNA showed an increase in C-terminal fragments (CTF) of APP (FIG. 5). Primary rat neurons were infected with APP695, a vector to express human APP. They were superinfected with a herpes vector expressing no shRNA, the APP-BP1 shRNA, or a random sequence irrelevant shRNA (missense). Cell lysates were analyzed by SDS-PAGE, blotted, and the blot probed with antibody 369, a rabbit polyclonal antibody raised against amino acids 645-694 of APP695 (gift from S. Gandy). The APP-BP1 shRNA increased the amount of APP C-terminal fragments. As a positive control, cells uninfected with the APP695 vector were treated with the gamma-secretase inhibitor L685459. Gamma-secretase cleaves APP to generate Aβ. Increases in APP C-terminal fragment are associated with increases in Aβ.

In neuronal cultures, it has been shown that synthetic Aβ provokes the neurons to undergo apoptosis (19, 17). Thus, reducing Aβ levels may reduce the symptoms of Alzheimer's disease.

### CONCLUSION

These data show that expression of two proteins in neurons—APP and APP-BP1—is specifically inhibited by shRNAs targeted to their transcripts and expressed from an HSV-1 vector. The data show that inhibition of APP-BP1 by shRNA can result in a strong increase in Aβ production, especially Aβ42, indicating that a major physiological function of APP-BP1 in neurons is to regulate APP processing. This finding that APP-BP1 regulates Aβ production or APP processing came as a surprise because we were focusing on a signaling hypothesis initiating from APP. Perhaps it should not be surprising, however, because APP-BP1 participates in protein turnover by activating neddylation. We have previously characterized an APP-BP1 binding protein, called ASPP2 (3), which partially inhibits neddylation and partially protects neurons from APP-BP1 overexpression-induced neuronal death. In addition, APP-BP1 is subject to several postranslational modifications, which may differentially modulate APP-BP1 regulation of Aβ genesis.

### REFERENCES

1. Bernstein, E., et al., 2001, Nature 409:363-366.
2. Brummelkamp, T. R., et al., 2002, Science 296:550-553.
3. Chen Y., et al., 2003, J. Neurochem. 85:801-809.
4. Clark, M. S., et al., 2002, J. Neuroscience 22:4550-4562.
5. Davison, A. J., et al., 1981, J. Gen. Virol. 55:315-331.
6. Elbashir, S M, et al., 2001, Nature 411:494-498.
7. Fire, A., et al., 1998, Nature 391:806-811.
8. Fraefel, C., et al., 1996, J. Virol. 70:7190-7197.
9. Geller, A. I., et al., 1990, Proc. Natl. Acad. Sci. USA 87:8950-8954.
10. Harborth, J., et al., 2003, Antisense Nucleic Acid Drug Dev. 13:83-105.
11. Johnston, K. M., et al., 1997, Human Gene Therapy 8:359-370.
12. Kawasaki, H., et al., 2003, Nucl. Acids Res. 31:700-707.
14. Lim, F., et al., 1996, BioTechniques 20:460-469.
15. Lim, F. et al., 1999, Current Protocols in Neuroscience 4.13.1-4.13.17.
16. Ling, X., et al., 2004, BioTechniques 36:450-460.
17. Loo, D., et al., 1993, Proc. Natl Acad. Sci. USA 90:7951-7955.
18. Martin, D. W., et al., 1991, J. Virol. 65:4359-4369.
19. Mattson M P. Apoptosis in neurodegenerative disorders. Nat. Rev. Mol. Cell Biol. 1:120-129.
20. Miller, V. M., et al. 2004, Nucl. Acids. Res. 32:661-668.
21. Neve, R. L., et al., 1999, Current Protocols in Neuroscience 4.12.1-4.12.7.
22. Paddison, P. J. et al., 2002, Genes Devel. 16:948-958.
23. Patterson, T., et al., 1990, J. Gen. Virol. 71:1775-1783.
24. Spaete, R. R., et al., 1985, Proc. Natl. Acad. Sci. USA 82:694-698.
25. Spankuch, B., et al., 2004, J. Natl. Cancer Inst. 96:862-872.
26. Wang, S., et al., 1996, J. Virol. 70:8422-8430.
26b. Wong, S. W. et al., 1991, J. Virol. 2601-2611.
27. Xiao-dong, L., et al., 2004, Biochem. Biophys. Res. Commun. 324:1173-1178.
28. Yu, J. Y., et al., 2003, Mol. Ther. 7:228-236.
29. Zamore, P. D., et al., 2000, Cell 101:25-33.
30. Fish, R. J. et al., 2004, BMC Molecular Biology 5:9.
31. Chen, Y. Z., 2004, Apoptosis 9:415-422.
32. Mullan, M., 1992, Nature Genet. 1:345-347.
33. Lee, V. M. et al., 2001, Ann. Rev. Neurosci. 24:1121-1159.
34. Lewis, J. et al., 2001, Science 293:1487-91.
35. Oddo, S. et al., 2003, Neuron 39:409-421.
36. Chen, Y. et al., 2003, J. Cell Biol. 163:27-33.
37. Chen, Y. et al., 2000, J. Biol. Chem. 275:8929-35.
38. Wang, Y. et al., 2000, Am. J. Physiol. Cell Physiol. 278:C519-C626.

All patents, patent applications, and other references cited herein are hereby incorporated by reference.

What is claimed is:

1. A recombinant nucleic acid molecule comprising:
(a) a herpes virus packaging signal sequence;
(b) a herpes virus origin of replication;
(c) a segment expressing a light-emitting marker; and
(d) a transfer RNA promoter upstream of a restriction endonuclease recognition sequence.

2. The recombinant nucleic acid molecule of claim 1 wherein the transfer RNA promoter is within 100 bp upstream of the restriction endonuclease recognition sequence.

3. The recombinant nucleic acid molecule of claim 1 wherein the transfer RNA promoter is within 10 bp upstream of the restriction endonuclease recognition sequence.

4. The recombinant nucleic acid molecule of claim 1 that comprises SEQ ID NO:1.

5. The recombinant nucleic acid molecule of claim 1 that consists of SEQ ID NO:1.

6. A recombinant nucleic acid molecule comprising:
(a) a herpes virus packaging signal sequence;
(b) a herpes virus origin of replication;
(c) a segment expressing a light-emitting marker; and
(d) a transfer RNA promoter linked to (e) a segment encoding a short hairpin RNA (shRNA) that is adapted to degrade in vivo to a small interference RNA (siRNA) that is complementary to a segment of a target gene.

7. The recombinant nucleic acid molecule of claim 6 wherein the origin of replication comprises nucleotides 6651-6849 of SEQ ID NO:1, or the complement thereof.

8. The recombinant nucleic acid molecule of claim 7 wherein the origin of replication comprises nucleotides 6334-7107 of SEQ ID NO:1, or the complement thereof.

9. The recombinant nucleic acid molecule of claim 6 wherein the packaging signal sequence is an HSV-1 packaging signal sequence.

10. The recombinant nucleic acid molecule of claim 9 wherein the packaging signal sequence comprises SEQ ID NO:2, or the complement thereof.

11. The recombinant nucleic acid molecule of claim 10 wherein the recombinant nucleic acid molecule comprises nucleotides 3011-4021 of SEQ ID NO:1, or the complement thereof.

12. The recombinant nucleic acid molecule of claim 6 wherein the transfer RNA promoter is a tRNA^{val} promoter.

13. The recombinant nucleic acid molecule of claim 12 wherein the tRNA^{val} promoter comprises nucleotides 7-113 of SEQ ID NO:1, or the complement thereof.

14. The recombinant nucleic acid molecule of claim 6 wherein the light-emitting marker is green fluorescent protein (GFP).

15. The recombinant nucleic acid molecule of claim 6 wherein the molecule is smaller than 15 kb.

16. The recombinant nucleic acid molecule of claim 6 wherein the molecule is at least 15 kb in size.

17. The recombinant nucleic acid molecule of claim 6 wherein the shRNA forms a stem-loop structure having a stem of 19 to 29 base pairs.

18. The recombinant nucleic acid molecule of claim 6 comprising at least two adeno-associated virus inverted terminal repeat sequences.

19. Herpes virus particles comprising the recombinant nucleic acid molecule of claim 6.

20. The virus particles of claim 19 wherein the virus particles are HSV-1 particles.

21. A method of inhibiting expression of a target gene in cells comprising:
(i) transforming the cells with a recombinant nucleic acid molecule comprising:
(a) a herpes virus packaging signal sequence;
(b) a herpes virus origin of replication;
(c) a segment expressing a light-emitting marker; and
(d) a transfer RNA promoter linked to (e) a segment encoding a short hairpin RNA (shRNA) that is adapted to degrade in vivo to a small interference RNA (siRNA) that is complementary to a segment of a target gene; and
(ii) expressing the shRNA in the cells.

22. The method of claim 21 wherein the cells are neuronal cells.

23. The method of claim 21 wherein the cells are transformed in vivo in a mammal.

24. The method of claim 21 wherein the cells are transformed in vitro.

25. The method of claim 21 wherein the shRNA is expressed in vivo in a mammal to inhibit expression of the target gene.

26. The method of claim 21 wherein the recombinant nucleic acid to transform the cells is encased in herpes virus capsid proteins to form herpes virus particles, and the cells are transformed with the virus particles.

27. The method of claim 26 wherein the cells are transformed with a known quantity of the virus particles, wherein the quantity is determined by titrating the virus particles by transforming cells with the virus particles and measuring light emitted from the transformed cells by the visible marker.

28. The method of claim 21 wherein the target gene is an APP or APP-BP1 gene.

29. The method of claim 21 wherein the target gene is an APP, APP-BP1, or tau gene.

30. The method of claim 29 wherein the target gene is a mutant form of an APP gene or a tau gene associated with Alzheimer's disease.

31. The method of claim 30 wherein the target gene is APPsw or tauV337M.

32. A cell comprising the recombinant nucleic acid molecule of claim 6.
